(19) United States
(12) Patent Application Publication
(10) Pub. No.: US 2008/0056998 A1
(43) Pub. Date: Mar. 6, 2008

(54) COMPOSITION AND PROCESSES FOR CROSSING THE BLOOD-BRAIN BARRIER WITH POLYPEPTIDES FOR IN VIVO NEUROIMAGING

(75) Inventors: Pham Wellington, Toledo, OH (US); Anna Moore, Dracut, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/782,222

(22) Filed: Jul. 24, 2007

Related U.S. Application Data
(60) Provisional application No. 60/833,111, filed on Jul. 24, 2006.

(51) Int. Cl. A61B 5/00

(52) U.S. Cl. 424/9.6

(57) ABSTRACT

According to an exemplary embodiment of the present invention, a versatile delivery module based on a myristoylated polyarginine backbone can be provided to cross a blood-brain barrier (“BBB”). An incorporation of a fatty acid group can be achieved using a Schotten-Bauman reaction with quantitative yield, and a peptide can be further synthesized by conventional solid phase peptide synthesis (“SPPS”). An in vivo distribution of the delivery module into a brain over time using near-infrared (“NIR”) fluorescence imaging can be obtained. A fluorescent cargo can be detected in vivo after an intravenous injection and can be further characterized in perfused brains. A staining of an excised brain can show that the delivery module can primarily accumulate in neurons with occasional localization in astrocytes and endothelial cells. This exemplary approach can be used for the delivery of imaging probes and targeted therapeutics across the BBB.

### CROSS REFERENCE TO RELATED APPLICATIONS

This application claims priority from U.S. Patent Application Ser. No. 60/833,111, filed Jul. 24, 2006, the entire disclosure of which is incorporated herein by reference.

### STATEMENT REGARDING FEDERALLY SPONSORED RESEARCH

The invention was made with the U.S. Government support, at least in part, by National Institute of Health, Grant numbers R01-NS37074, R01-NS38731, R01-NS40529, R01-HL39810, P50-NS10828 and RR1407506. Thus, the U.S. government may have certain rights in the invention.

### FIELD OF THE INVENTION

The present invention relates to compositions and processes associated with the treatment and detection of neurological diseases. In particular, the present invention relates treatment and detection of neurological diseases by delivering therapeutic, diagnostic and/or imaging agents across a blood brain barrier of mammals.

### BACKGROUND OF THE INVENTION

It is recognized that an in vivo application of potentially useful therapeutic or imaging agents for the treatment or detection of neurological diseases can be hampered by the limited access of probes to a person's central nervous system across the blood brain barrier (“BBB”), which comprises tight junctions of brain capillaries associated with pericytes, and astrocyte foot processes. As basic neurological research continues to reveal novel targets for therapy, it has become increasingly important to deliver therapeutic agents across the BBB to the central nervous system. Previously, this delivery could be achieved by highly invasive direct injection or permeabilization of tight junctions using either an osmotic disruption with, for example, mannitol, or a biochemical opening with, for example, RMP-7 Alkermes or histamine. However, the highly invasive and disruptive nature of these methods have led to more desirable developments.

Certain recent advances over previous delivery methods have included developments of fatty acylated peptides for enhancing a membrane permeability. (See, e.g., Pham, W., Kircher, M. F., Weissleder, R., Tung, C. H., 2004. Enhancing membrane permeability by fatty acylation of oligoarginine peptides. Chembiochemistry 5, 1148-1151). The underlying exemplary principle allowing the fatty acylated peptides to enhance membrane permeability centers on the amphiphilic property brought about by the incorporation of a fatty acid analog with oligo-arginine peptides. A hydrophobic 14-carbon moiety of myristic acid, in combination with a polyarginine peptide, can demonstrate cellular internalization that is less invasive and disruptive. Further, as described in Galbiati, F., Guzzi, F., Magee, A. I., Milligan, G., Parenti, M., 1996. Chemical inhibition of myristoylation of the G-protein Gi1 alpha by 2-hydroxymyristate does not interfere with its palmitoylation or membrane association. Evidence that palmitoylation, but not myristoylation, regulates membrane attachment. Biochem. J. 313 (Pt. 3), 717-720 and Pham, W., Kircher, M. F., Weissleder, R., Tung, C. H., 2004. Enhancing membrane permeability by fatty acylation of oligoarginine peptides. Chembiochemistry 5, 1148-1151 the fatty acid analog can provide sufficient energy to anchor a peptide to a biological membrane without a permanent binding, as can be observed in the case of the 16-carbon palmitic acid. In addition, as described in Pham, W., Kircher, M. F., Weissleder, R., Tung, C. H., 2004. Enhancing membrane permeability by fatty acylation of oligoarginine peptides. Chembiochemistry 5, 1148-1151, the fatty acid-peptide formation can cross a cellular membrane of live cells efficiently and return to the cytoplasm with no registered toxicity. If provided, it may be desirably for the delivery modules to provide targeting molecules across the BBB to their respective active sites. In addition, it may be advantageous for the bioavailability of these delivered agents to be monitored over time using exemplary non-invasive imaging techniques.

Accordingly, there may be a need to overcome the deficiencies described herein above.

### SUMMARY OF THE INVENTION

To address and/or overcome the above-described problems and/or deficiencies as well as other deficiencies, exemplary embodiments of a composition according to the present invention can be provided including at least one fatty acylated peptide, which are configured to cross a blood-brain barrier of a mammal after an intravenous injection of the composition into the mammal.

In one exemplary embodiment of the composition according to the present invention, the fatty acylated peptide can include a dye. For example, the dye can be detected by an near-infrared fluorescence imaging procedure and/or system. In a further exemplary embodiment of the present invention, the fatty acylated peptide can include a therapeutic agent. In a still further exemplary embodiment of the present invention, the fatty acylated peptide can include both a dye and a therapeutic agent. In another exemplary embodiment of the present invention, the fatty acylated peptide can include a chelate and/or a therapeutic agent.

For example, according to one exemplary variant, the fatty acylated peptide can have the formula C14-βAla-(Arg)₇-Cys(R)—NH₂, in which, for example, R can be a dye and/or a therapeutic agent. If R is a dye, for example, the fatty acylated peptide can have the formula as follows: C14-βAla-(Arg)₇-Cys(Cy5.5)-NH₂.

In a further exemplary embodiment of the present invention, the composition can be localized in particular cells in the brain.

In addition, further exemplary embodiments of a method of monitoring a composition including at least one fatty acylated peptide can be provided. For example, the combination, intravenously injected into a mammal. Then, the mammal, may be imaged, can be prepared, and the fatty acylated peptide can include a dye and the dye can be detected by near-infrared fluorescence imaging. In a still further exemplary embodiment of the present invention, the mammal can be merged by a near-infrared fluorescence imaging procedure and/or system. For example, the exemplary method can also include the monitoring of the presence of the combination in the mammal over a period of time. In a further exemplary embodiment of the present invention, the fatty acylated peptide can include a chelate, and the chelate can be detected by magnetic resonance imaging procedure.

### BRIEF DESCRIPTION OF THE DRAWINGS

The detailed description below will refer to the following illustrations, wherein like numerals refer to like elements, and wherein:

FIG. 1 is an exemplary formula illustrating a synthesis of an exemplary MPAP-Cy5.5 delivery module;

FIG. 2 is three exemplary in vivo images of a mouse at a series of time periods after being intravenously injected with the exemplary MPAP-Cy5.5 delivery module;

FIG. 3 is three exemplary in vivo images of a mouse at a series of time periods after being injected with Cy5.5 dye alone;

FIG. 4 is three exemplary in vivo images of a mouse brain at a certain time after being intravenously injected with the exemplary MPAP-Cy5.5 delivery module;

FIG. 5 is three exemplary ex vivo images of a perfuse mouse brain at certain time after being intravenously injected with the exemplary MPAP-Cy5.5 delivery module, the Cy5.5 dye, and saline;

FIG. 6 is four exemplary ex vivo images of a coronal mouse brain slices obtained perfused mouse brain obtained certain time after the mouse was intravenously injected with the exemplary MPAP-Cy5.5 delivery module;

FIG. 7 is an exemplary graphical illustration of an HPLC chromatogram of a perfused mouse brain homogenate, with an inset of an absorbance spectrum of the homogenate co-registered through a built-in fluorescence detector matched with that of a Cy5.5 dye;

FIG. 8 is an exemplary image of a coronal brain section identifying a cortex, a hippocampus, and a thalamus;

FIG. 9 is exemplary dual channel fluorescence microscopy images of cell-specific distribution of the exemplary MPAP-Cy5.5 delivery module in a brain; and

FIG. 10 is exemplary confocal microscopy images of brain endothelium at a series of times after being intravenously injected with the exemplary MPAP-Cy5.5 delivery module.

### DETAILED DESCRIPTION OF EXEMPLARY EMBODIMENTS

According to one exemplary embodiment of the present invention, a delivery module may be used in a non-invasive and non-disruptive method for delivery across the BBB. For example, a myristoylated polyarginine peptide (MPAP) may be used as the delivery module. For the purpose of in vivo work, the delivery module can be further modified to accommodate a near-infrared (“NIR”) fluorescent dye so that the accumulation and distribution of the dyed delivery module can be monitored over time by optical imaging. The myristoyl moiety can act as a steering component to promote membrane association. Additionally, hydrophilic polycations of arginine analogs can enhance interactions of the delivery module with negative charges associated with the BBB cell membranes, which can originate from sialic acid and heparin sulfate residues on the surface of endothelial cells. These kinds of electrostatic interactions between cationic peptides and negative charges associated with the BBB cell membranes may mediate adsorptive endocytosis. (See, e.g., Drin, G., Rousselle, C., Scherrmann, J. M., Rees, A. R., Temsamani, J., 2002. Peptide delivery to the brain via adsorptive-mediated endocytosis: advances with SynB vectors. AAPS PharmSci. 4, E26; Vorbrodt, A. W., 1989. Ultracytochemical characterization of anionic sites in the wall of brain capillaries. J. Neurocytol. 18, 359-368.)

According to the exemplary embodiment of the present invention, the chemical synthesis and in vivo testing of the exemplary delivery module can illustrate show an accumulation of the exemplary delivery module in a mouse brain and these findings can be correlated with histological analyses.

Exemplary Incorporation of a Fatty Acid into a β-Alanine Linker

In an exemplary preparation of the delivery module, a fatty acid can be incorporated into a β-alanine linker. For example, solution phase chemistry may be employed in a Schotten-Baumann reaction to derivatize myristic acid into a N-terminal form of a β-alanine linker. In an exemplary preparation, 0.92 milliliters (mL) (3.38 millimoles (mmol)) of myristoyl chloride can be added drop-wise to a solution of β-alanine (602.25 milligrams (mg) (6.76 mmol)) in 0.3 Molar (M) NaOH held at a temperature not exceeding thirty (30) Degrees Celsius. Following the addition of myristoyl chloride, 20.0 mL of 5.0 M NaOH can be added to the solution, which should continue to be held at a temperature not exceeding 30 Degrees Celsius. The reaction can take place at an inter-phase between the β-alanine solution in sodium hydroxide (NaOH) and a fatty acyl chloride. (See, e.g., publication 5 identified below.) The subsequent solution can be stirred at room temperature for 1 hour. After stirring, the solution can be poured into a 1:1 mixture of 6 M HCl in water to yield a precipitate. A water-insoluble product of myristoylated β-alanine can be precipitated out quantitatively in 1 hour at room temperature. The precipitate can then be collected, washed with water, and recrystallized with methylene chloride and methanol to yield a colorless powder of myristoylated β-alanine.

Exemplary Solid Phase Peptide Synthesis

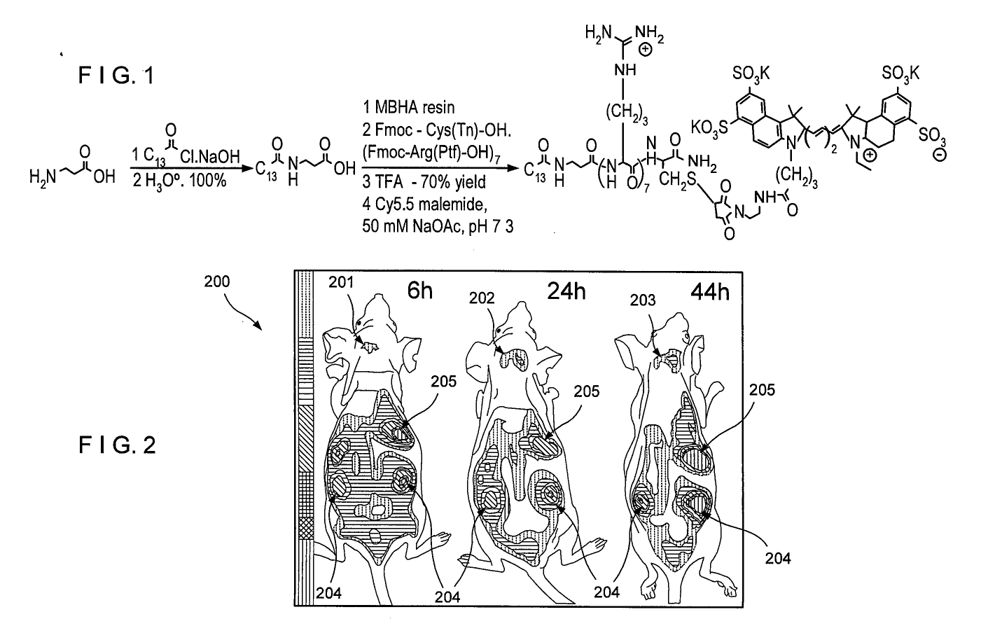

A peptide can be synthesized by a conventional solid phase peptide synthesis (“SPPS”) method involving Fmoc solid phase chemistry and purification of the peptide in a way that is commonly known in the art, as described in Pham, W., Kircher, M. F., Weissleder, R., Tung, C. H., 2004. Enhancing membrane permeability by fatty acylation of oligoarginine peptides. Chembiochemistry 5, 1148-1151. In an exemplary embodiment of the present invention, the following exemplary modifications to the conventional SPPS method can also yield peptides. In an exemplary modification to the conventional SPPS method, Fmoc β-alanine can be substituted with myristoylated β-alanine. Further, referring to FIG. 1, in an exemplary embodiment of the in vivo imaging applications, the delivery module can be conjugated to the NIR Cy5.5 dye. In the conjugation, a cysteine can be deployed on a MPAP carboxyl-terminal where the thiol moiety can react with a commercially available Cy5.5 maleimide via a Michael addition reaction to form a MPAP-Cy5.5. In an exemplary synthesis, conjugation of C14-βAla-(Arg)₇-Cys(SH)—NH₂ with Cy5.5 maleimide, from such place as, e.g., Amersham Biosciences, Piscataway, N.J., can be completed in 50 millimolar (mM) NaOAc, having a pH of 7.3, for 45 min at room temperature to yield an exemplary delivery module of C14-βAla-(Arg)₇-Cys(Cy5.5)-NH₂, designated as MPAP-Cy5.5. This strategy can provide a superior yield compared to the conjugation of a fatty acid group to a peptide on solid support where steric hindrance as well as low solubility of the fatty acid analog in DMF are unproductive.

The resulting product may be examined using, for example, MALDI mass spectroscopy and can show about 2537.04 calculated versus 2537.16 found, which may be attributed to (M+H)⁺. The yield of the MPPS may be measured by weighing the resulting product and converting the mass into mmol using the following equation:
(mmol of fatty acylated peptide/0.1 mmol resin)×100

Exemplary Animals

In the exemplary embodiment of the present invention, the delivery module can be injected into various mammals having a BBB. For example, the delivery module can be injected into mice.

Exemplary NIR Fluorescence Imaging

In an exemplary embodiment of the present invention, the delivery of the delivery module into a brain may be demonstrated using, for example, in vivo NIR fluorescence imaging experiments. In the exemplary embodiment of the present invention, for in vivo optical imaging, n mice can be injected intravenously with about 2.5 nmol of the exemplary delivery module MPAP-Cy5.5, Cy5.5 alone, or saline solution. In an exemplary embodiment of the present invention, n=4 for each group of solutions for a total of 12 mice. Imaging can be performed at various times after injection. For example, it may be performed about 15 and 30 minutes after injection, as well as 1, 2, 3, 6, 24 and 44 hours after injection. For exemplary NIR fluorescent imaging, the mice can be placed into an imaging system, for example a Kodak Imaging Station IS2000MM, equipped with a band-pass filter at about 630 nm and a long pass filter at about 700 nm, such as the Chroma Technology Corporation. Exemplary images can be captured with e.g., charge-coupled device (“CCD”) camera embedded in the imaging system and exemplary analyses may be performed using Kodak ID3.6.3 imaging software.

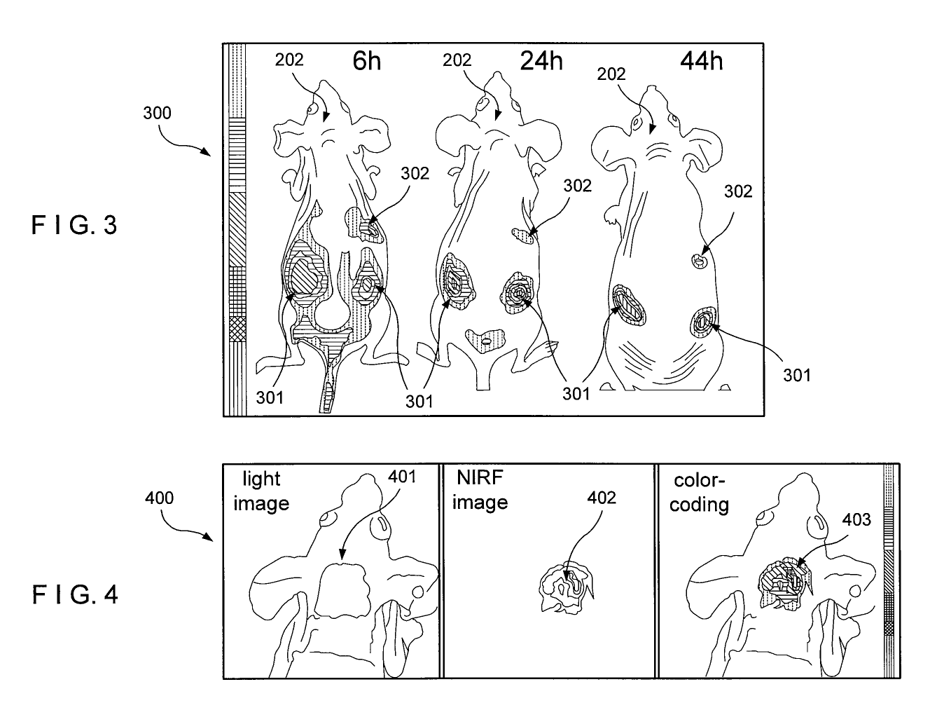

Initially, after an exemplary intravenous injection of the delivery module into an mammal, such as a mouse, there may be no defined fluorescent signal in the brain for a few hours. Referring to FIG. 2, however, a progressively increasing NIR fluorescent signal in the head summit 200 can occur at 6 hours post-injection 201, thereby detecting the presence of the delivery module. An increase in the emitted light can occur 24 hours after injection 202, and the signal can continue to accumulate up to approximately 2 days or more 203. Referring to FIG. 3, it can be observed that no signal appears for brains from control animals treated with Cy5.5 or saline during the same period of time. Following the systemic distribution of the delivery module, there can be an accumulation of the delivery module in peripheral tissues, such as the kidneys 204 and the spleen 205, followed by eventual clearance. Similar accumulation in the mouse body 301 302 can be observed after the injection of Cy5.5 dye alone, and no accumulation in the brain 303 can be observed at any point in time. Referring to FIG. 4, which show an exemplary light image 401, NIR image 402 and pseudo-color image 403 of a mouse brain at about 24 hours post-injection 400 of the exemplary delivery module MPAP-Cy5.5, the delivery module can be observed to have crossed the BBB and accumulate within the mouse brain.

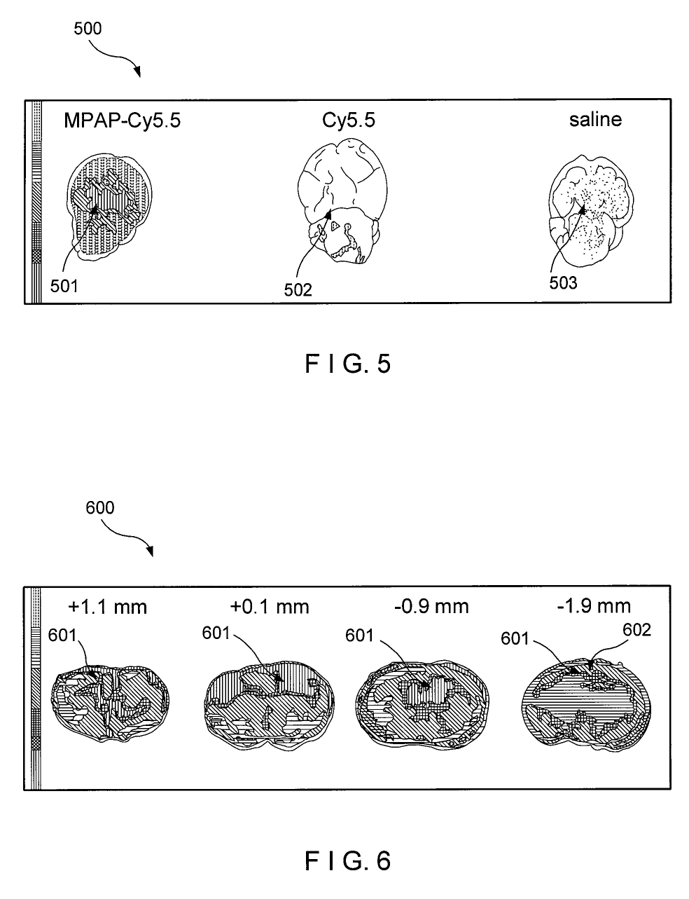

In another exemplary embodiment of the present invention, fluorescence signals may be observed using exemplary ex vivo imaging performed on the exemplary mouse brains post injection of the exemplary delivery module. Exemplary Ex vivo NIR fluorescence imaging can be performed, e.g., 24 hours after the injection of the exemplary MPAP-Cy5.5 delivery module. Upon sacrificing the mice, each mouse brain can be removed, placed into the exemplary imaging system, and imaged as described above. Referring to FIG. 5, a strong NIR fluorescent signal can be observed from the exemplary mouse brain affected by the exemplary MPAP-Cy5.5 delivery module 501. Further, the brains from the control mice treated with Cy5.5 502 or saline 503 alone likely have no significant signal beyond background levels.

Additional observations regarding accumulation of the exemplary delivery module in the brain can be obtained after imaging 1 mm-thick coronal brain slices from the exemplary mouse brain. The exemplary mouse brain can be cut into 1 mm coronal brain sections using a slicing apparatus, such as the RBM-2000C provided by Analytical Scientific Instruments, taken at the following exemplary anteroposterior coordinates from bregma, e.g.: +1.1 mm, +0.1 mm, −0.9 mm, −1.9 mm. Referring to FIG. 6, the exemplary brain slices can then be placed into the exemplary imaging system and imaged 600 as described above. The exemplary delivery module can be distributed randomly throughout the brain parenchyma 601 including the hippocampus as seen in the −1.9 mm slice 602.

Exemplary HPLC Study of a Perfused Brain

It may be desirous to show that the observed fluorescent signal from the NIR is actually from the intact delivery module accumulated in the brain and not from a partial Cy5.5-associated peptide that has been metabolized during systemic transport. In a further exemplary embodiment of the present invention, the mouse brains can be perfused for 24 hours after injection of the delivery module in order to remove intravascular contents. The brain homogenates can then be subjected to exemplary High-Powered Liquid Chromatography (“HPLC”) analysis.

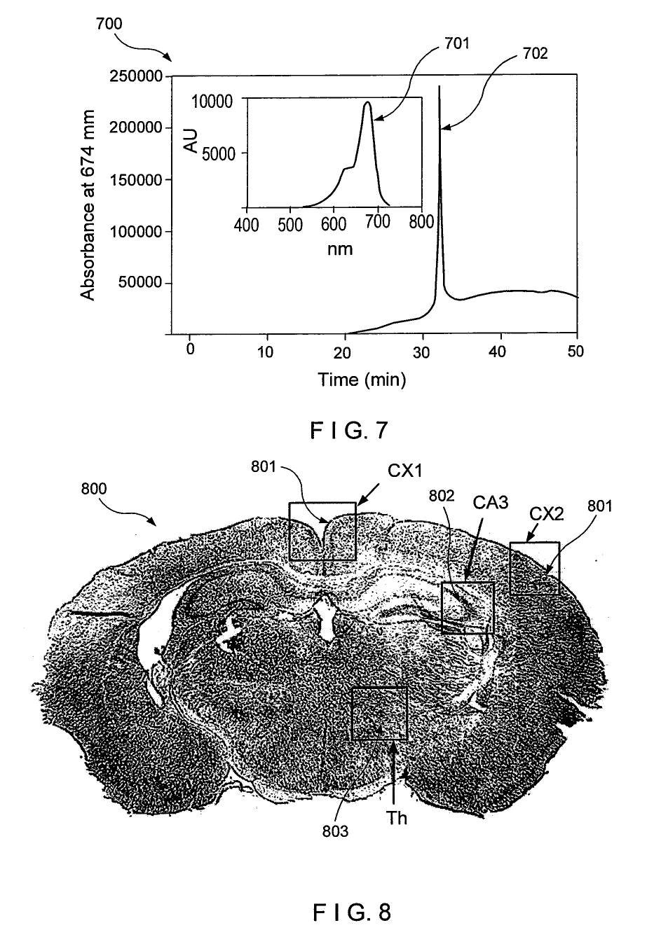

According to an exemplary embodiment of the present invention, n mice (n=4 for example) can be injected intravenously with the delivery module. The mouse brain uptake of the compound can be detected by HPLC after in situ brain perfusion. A perfused brain can be soaked in a HPLC mobile phase solution for 15 minutes and homogenized on ice using a mechanical homogenizer, such as that which is marketed by Virtis in Gardiner, N.Y. A resulting brain residue can be filtered using, for example, 0.45 Am Whatman Nylon filters, lyophilized, and analyzed by 2D-HPLC, such as that whish is marketed by Lachrome Elite, Hitachi in San Jose, Calif. In an exemplary detection of an intact exemplary delivery module, an exemplary absorption wavelength of 760 nanometers (nm) for Cy5.5 dye can be selected for comparison. The intact exemplary delivery module can have an HPLC peak associated with it. This peak can be compared with the retention time of an authentic sample. In addition, the detected peak can be correlated and matched with the absorbance spectrum profile of Cy5.5 dye. Referring to FIG. 7, the results of this exemplary study 700 can show that the retention time can be 32 minutes 701, which matches the retention time that can be shown for the exemplary MPAP-Cy5.5 delivery module without being injected. Furthermore, the absorbance profile of this peak can correspond to the absorbance profile of Cy5.5 dye at approximately 670 nm 702.

Upon switching the detection wavelength to 220 nm, the retention time peak at 32 min can be detected along with other more polar peaks, such as those associated with brain metabolites. Taken together, these data can demonstrate that the delivery module may likely not degrade in systemic circulation and indeed can accumulate in the brain parenchyma.

Exemplary Dual-Channel Fluorescence Microscopy and Confocal Microscopy

When the exemplary MPAP-Cy5.5 delivery module penetrates the BBB, exemplary dual-channel fluorescence microscopy can be used to observe whether the distribution of the exemplary MPAP-Cy5.5 delivery module in the exemplary brain forms in a cell-type-specific manner. In a further exemplary embodiment of the present invention, an exemplary distribution of the exemplary MPAP-Cy5.5 delivery module within different cell types of a brain can be determined with dual-channel fluorescence microscopy. For example, cryopreserved serial brain sections from mice perfused with the MPAP-Cy5.5 delivery module can be stained for neurons, astrocytes, and endothelial cells using anti-neuronal nuclei antibody (such as, for example, NeuN, monoclonal, from Chemicon International, Temecula, Calif.), anti-glial fibrillary acidic protein antibody (such as, for example, GFAP from Chemicon International, Temecula, Calif.), and anti-CD31 (such as, for example PECAM-1 from BD Biosciences Pharmingen, San Jose, Calif.). The brain sections may be further incubated with corresponding FITC-conjugated secondary antibodies. Further, slides of the brain sections can be mounted in a mounting medium, such as, for example, a Vectashield mounting medium from Vector Laboratories in Burlingame, Calif. Dual-channel fluorescence microscopy in, for example, the Cy5.5 and FITC channels can be performed using, for example, a Nikon Eclipse 50i fluorescence microscope equipped with an appropriate filter set, such as, for example, one provided by Chroma Technology Corporation in Rockingham, Vt. Exemplary images can be acquired using a CCD camera with near-infrared sensitivity (such as, for example, a SPOT 7.4 Slider RTKE from Diagnostic Instruments in Sterling Heights, Mich.). FIG. 8 shows an exemplary picture of a cross-section of an exemplary coronal brain section 800, which identifies the cortex 801, the hippocampus 802, and the thalamus 803. Referring to FIG. 8, the exemplary MPAP-Cy5.5 delivery module can be detected in almost all regions of the brain, including the cortex 801, the hippocampus 802, and the thalamus 803.

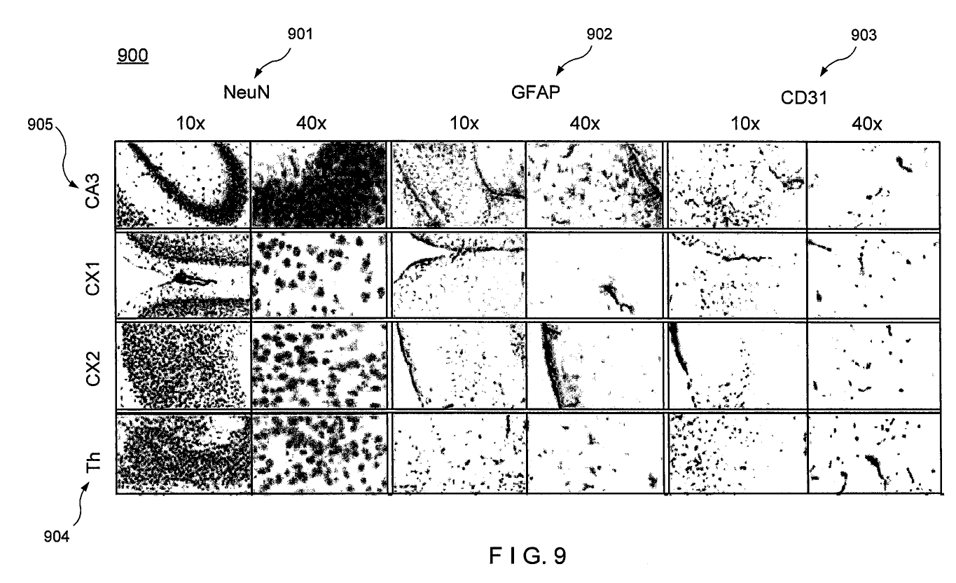

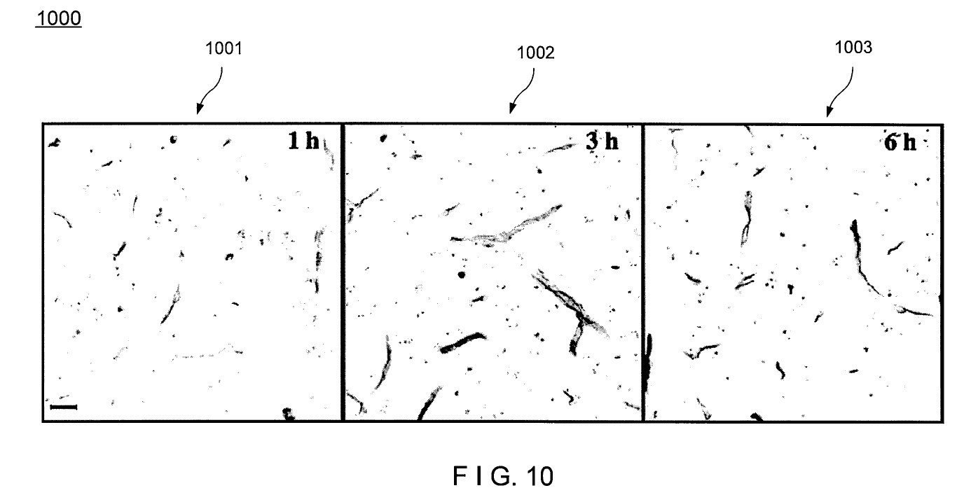

FIG. 9 shows exemplary images taken of a dual channel fluorescence microscope of cell-specific distribution of the exemplary MPAP-Cy5.5 delivery module in the brain 900. Referring to FIG. 9, the exemplary MPAP-Cy5.5 delivery module can primarily accumulate in neurons 901. This accumulation may be predominantly localized within the cell cytoplasm. Although a mechanism for neuronal accumulation remains unclear, it may be possible that preferential targeting into neurons can be related to the higher lypophilicity of neuronal membranes as compared to other cell types. (See, e.g., publications 4 and 6.) Lypophilic peptides may have an advantage to penetrate by adsorptive endocytosis. (See, e.g., publications 2.) In contrast, as can be observed in FIG. 9, astrocytes 902 may not show widespread accumulation of the exemplary MPAP-Cy5.5 delivery module. However, some signal may be detectable in the thalamus 904 and the CA3 area of the hippocampus 905. Similarly, endothelial cells 903 may exhibit no significant localization of the exemplary MPAP-Cy5.5 delivery module, even though these would be the initial cells seen by the exemplary MPAP-Cy5.5 delivery module after injection.

Further, fluorescence confocal microscopy techniques/systems can be employed to investigate a delivery route of the delivery module after injection. For example, mammals can be injected intravenously with about 2.5 nmol of the exemplary delivery module MPAPCy5.5, perfused, and sacrificed 1, 3 or 6 hours after injection. Brain sections from the sacrificed animals may be frozen. The frozen brain sections can then be stained with, for example, anti-CD31 antibodies followed by FITC-conjugated secondary antibody as described above. Dual-channel confocal microscopy can be performed, for example, in the FITC and Cy5.5 channels. Further, confocal microscopy can be performed using, for example, an Axiovert 200 M inverted microscope (from, e.g., Carl Zeiss, Inc. Thornwood, N.Y.), equipped with an LSM Pascal Vario RGB Laser Module (Arg 458/488/514 nm, HeNe 543 nm, HeNe 633 nm). An exemplary confocal images with a slice thickness of, for example, <0.9 Am can be acquired to produce, for example, FITC and Cy5.5 staining as well as Nomarski optics. Exemplary summation projection of all background-corrected confocal slices can be produced using, for example, LSM 5 Pascal Software. Further, the exemplary confocal images can be color-coded with, for example, green for FITC and red for Cy5.5. As can be observed in FIG. 10, confocal microscopy studies images 1000 can reveal that the delivery module may pass through the endothelium within 1 hour after injection 1001 and increases in hours 3 1002 and 6 1003. Complete transcytosis of the exemplary MPAP-Cy5.5 delivery module may occur during the first hour, however, the detailed kinetics of the dynamic effect may require additional investigation.

For transcytosis of peptides through the BBB, an exemplary embodiment of the present invention can include three exemplary steps: (a) binding and internalization at a luminal side of the endothelial cell membrane, (b) diffusion through the cytoplasm of endothelia cells, and (c) externalization at the basolateral side of the endothelial cells. (See, e.g., publication 1.) At the time when the exemplary MPAP-Cy5.5 delivery module accumulation can be detected in vivo, such as approximately at hour 6 as shown in FIG. 2, most of the exemplary MPAP-Cy5.5 delivery module may have been already externalized from the endothelium into the brain parenchyma.

According to a further exemplary embodiment of the present invention the delivery module can be derivatized with lanthanide chelates for the magnetic resonance imaging (“MRI”) techniques and systems. If a sufficient in vivo concentration can be achieved, the derivitized exemplary delivery module may provide anatomical and physiological parameters of the delivery concomitantly, similar to what is described in Massoud, T., Gambhir, S., 2003. Molecular imaging in living subjects: seeing fundamental biological processes in a new light. Genes Dev. 17, 545-580.

In another exemplary embodiment of the present invention, the delivery module may penetrate the BBB after intravenous injections, may be easily visualized via optical imaging in vivo, and may also be primarily targeted into neurons. For example, 6 hours after intravenous injection, the in vivo imaging may detect the delivery module within the brain. Because the kinetics of first pass accumulation in liver, kidneys and spleen may be much faster, it may be possible that the delivery module accumulates via secondary passes into the brain. Alternatively, the accumulation may begin immediately after injection, but the NIR fluorescent signal may not be strong enough to be detected through the skull until the delivery module accumulates in higher concentration. It may be desirable to substitute the Cy5.5 dye with an in-house-developed dye, as described in publication 9 identified below, which may have better optical properties for in vivo imaging and stability, which may increase tissue penetration. In addition, issues such as tissue scattering and limited depth penetration, as well as low spatial resolution and inadequate capacity for quantitative estimation of delivery module accumulation may need to be addressed in order to use in vivo optical imaging to detect and characterize discrete events such as imaging of isolated regions of interest within an internal organ.

Thus, an exemplary embodiment of the present invention can include a non-invasive and nondisruptive strategy for delivering compounds across the BBB based on a delivery module, such as a myristoylated polyarginine peptide (MPAP). The delivery module can be loaded with a therapeutic and/or imaging cargo, the combination of which can be subsequently delivered to a brain for therapy, treatment, and/or imaging. For example, the delivery module can be conjugated with an optically detectable dye. Given the time and cost efficiency of optical imaging, this approach may facilitate the delivery of BBB-penetrating compounds for the detection and potential treatment of brain cancer and other central nervous system disorders as well as for the screening of new drugs, including the staging of therapeutic effects in mouse models of neurodegenerative diseases.

Although the present invention has been described with respect to particular embodiments thereof, variations are possible. The present invention may be embodied in specific forms without departing from the essential sprit or attributes thereof. For example, although the present invention is illustrated with embodiments having a dye detectable by FIR imaging, other dyes may be used. Additionally, although the present invention is illustrated with β-Alanine and myristic acid, other peptides and fatty acids may be used to form a fatty acylated peptide. Further, other forms of imaging may be used to detect the presence of the fatty acylated peptide in the brain. It is desired that the embodiments described herein be considered in all respect illustrative and not restrictive and that reference be made to the appended claims and their equivalents for determining the scope of the invention.

What is claimed is:

1. A composition, comprising:
at least one fatty acylated peptide, the composition cross, a blood barrier of a brain, of at least one of an animal or a human upon application thereof.

2. The composition according to claim 1, wherein the at least one fatty acylated peptide comprises a dye.

3. The composition according to claim 2, wherein the dye is detectable by a near-infrared fluorescence imaging procedure.

4. The composition according to claim 2, wherein the at least one fatty acylated peptide further comprises a therapeutic agent.

5. The composition according to claim 1, wherein the at least one fatty acylated peptide comprises a therapeutic agent.

6. The composition according to claim 1, wherein the at least one fatty acylated peptide comprises a chelate.

7. The composition according to claim 1, wherein the at least one fatty acylated peptide is formulated as C14-βAla-(Arg)₇-Cys(R)—NH₂.

8. The composition according to claim 7, wherein R is a dye.

9. The composition according to claim 7, wherein R is a therapeutic agent.

10. The composition according to claim 7, wherein R is a chelate.

11. The composition according to claim 1, wherein at least one fatty acylated peptide causes the composition to be localized in particular cells in the brain.

12. A method of providing a composition including at least one fatty acylated peptide, comprising:
the combination into at least one of an animal or a human; and
imaging at least a portion of the mammal, wherein the composition cross a blood barrier of the brain of at least one of the animal or the human.

13. The method of claim 12, wherein the at least one fatty acylated peptide comprises a dye.

14. The method of claim 13, wherein the dye is detectable by a near-infrared fluorescence imaging procedure.

15. The method of claim 12, wherein the imaging step is performed using a near-infrared fluorescence imaging procedure.

16. The method of claim 12, further comprising monitoring a presence of the combination in the portion over a period of time.

17. The method of claim 12, wherein the at least one fatty acylated peptide further comprises a therapeutic agent.

18. The method of claim 12, wherein the at least one fatty acylated peptide comprises a dye.

19. The method of claim 18, wherein the imaging step is performed using a magnetic resonance imaging procedure.
